(19) United States
(12) Patent Application Publication
(10) Pub. No.: US 2025/0222204 A1
(43) Pub. Date: Jul. 10, 2025

(54) Injector

(71) Applicant: HALOZYME, INC., SAN DIEGO, CA (US)

(72) Inventor: Kevin Ruda, Hopkins, MN (US)

(73) Assignee: HALOZYME, INC., SAN DIEGO, CA (US)

(21) Appl. No.: 19/006,459

(22) Filed: Dec. 31, 2024

Related U.S. Application Data
(60) Provisional application No. 63/619,669, filed on Jan. 10, 2024.

(51) Int. Cl. A61M 5/20; A61M 5/32

(52) U.S. Cl. CPC A61M 5/2033; A61M 5/322; A61M 2005/206

(57) ABSTRACT

An injector includes a housing extending along a longitudinal axis, the housing has a proximal end and a distal end. A medicament container defines a volume therein. A needle is coupled to and extending from a distal end of the medicament container. An energy system has a first energy source and a second energy source. The first energy source is configured to be activated before the second energy source.

### CROSS-REFERENCE TO RELATED APPLICATIONS

This application claims the benefit of U.S. Provisional Application No. 63/619,669 filed Jan. 10, 2024, entitled “Injector”, which is incorporated by reference herein in its entirety.

### TECHNICAL FIELD

The present disclosure generally relates to injectors and, in some embodiments, to an injector having a two-stage energy source.

### SUMMARY

An injector may include a housing extending along a longitudinal axis, the housing having a proximal end and a distal end; a medicament container defining a volume therein; a needle coupled to and extending from a distal end of the medicament container; and an energy system having a first energy source and a second energy source, wherein the first energy source is configured to be activated before the second energy source.

In certain embodiments, the injector further includes a first ram engageable with the first energy source and a second ram engageable with the second energy source. In certain embodiments, movement of the first ram moves the second ram relative to the housing. In certain embodiments, the second ram is detachably coupled to the first ram, and the second ram further includes a flexible arm extending therefrom, the flexible arm releasably engageable with the first ram.

In certain embodiments, the medicament container includes a flange extending radially outward from a proximal end. In certain embodiments, activation of the first energy source and movement of the first ram along the longitudinal axis relative to the housing causes engagement of the flexible arm and the flange. In certain embodiments, the flange disengages the flexible arm from the first ram, thereby activating the second energy source.

In certain embodiments, the first ram includes a ram aperture extending therethrough configured to receive the flexible arm. In certain embodiments, the flexible arm includes an engagement feature extending radially therefrom, the engagement feature engageable with the ram aperture to releasably couple the second ram to the first ram. In certain embodiments, engagement of the flexible arm and the flange disengages the engagement feature of the flexible arm from the ram aperture.

In certain embodiments, the second energy source urges the second ram distally along the longitudinal axis relative to the medicament container. In certain embodiments, the second ram is movable along the longitudinal axis relative to the first ram.

In certain embodiments, the second ram is movable a first distance within the volume of the medicament container when the first energy source is activated and a second distance when the second energy source is activated. In certain embodiments, the first distance is greater than the second distance.

In certain embodiments, the first energy source engages the housing and the first ram. In certain embodiments, the second energy source engages the first ram and the second ram.

In certain embodiments, the second energy source is at least partially nested in the first energy source. In certain embodiments, the first energy source extends outside of the medicament container when activated and wherein the second energy source extends inside of the medicament container when activated.

In certain embodiments, the medicament container contains a volume of a medicament. In certain embodiments, the energy system is configured to eject the volume of the medicament from the medicament container in less than 30 seconds.

In certain embodiments, the first energy source and the second energy source are connected in series. In certain embodiments, the injector further includes a trigger mechanism configured to activate the first energy source.

### BRIEF DESCRIPTION OF THE DRAWINGS

The following detailed description of embodiments of the injector, will be better understood when read in conjunction with the appended drawings of an exemplary embodiment. It should be understood, however, that the invention is not limited to the precise arrangements and instrumentalities shown.

In the drawings:

FIG. 1 is a bottom perspective view of an injector in accordance with an exemplary embodiment of the present invention;

FIG. 2 is a front cross-sectional view of the injector of FIG. 1;

FIG. 3 is a partial front cross-sectional view of an energy system of the injector of FIG. 1;

FIG. 4 is a partial front cross-sectional view of an energy system of the injector of FIG. 1 shown after the first energy source has been activated and before the second energy source has been activated;

FIG. 5 is a top perspective view of a proximal end of a first ram of the energy system of the injector of FIG. 1;

FIG. 6 is a partial front cross-sectional view of a second ram of the energy system of the injector of FIG. 1 being activated; and

FIG. 7 is a partial front cross-sectional view of a second ram of the energy system of the injector of FIG. 1 shown after the first energy source has been activated and after the second energy source has been activated.

### DETAILED DESCRIPTION

Injectors play a crucial role in modern medicine by providing a reliable and efficient means of administering medications, particularly for self-administration or in emergency situations. Their importance lies in ensuring accurate dosage delivery, improving patient compliance, and facilitating the timely administration of life-saving treatments, thereby enhancing overall healthcare outcomes. Injectors commonly rely on energy sources such as mechanical springs, compressed gases, or electronic mechanisms to generate the force necessary for precise and controlled medication delivery. The benefit of springs lies in their ability to deliver a controlled and precise force during injection, ensuring accurate dosage and consistent delivery.

The duration of spring force in injectors, however, presents several challenges. One limitation is the need to balance the force's duration to accommodate varying viscosities of medications. Some drugs may require a slower injection to prevent pain or tissue damage, while others may benefit from a faster delivery for improved efficacy. Achieving this balance is paramount to ensure that the spring force maintains consistency throughout the injection, mitigating the risk of discomfort or complications for the user. Additionally, considerations such as viscosity changes over time or temperature variations further complicate the task of designing autoinjectors that deliver medications reliably and comfortably.

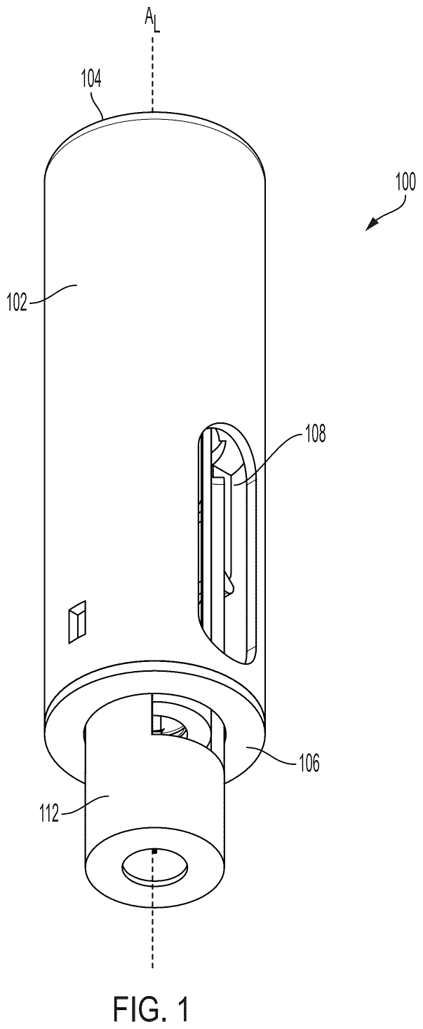

Referring to the drawings in detail, wherein like reference numerals indicate like elements throughout, there is shown in FIGS. 1-7 an injector, generally designated 100, in accordance with an exemplary embodiment of the present invention. Injector 100 may include a two-stage energy system 124 to ensure accurate and consistent dosing throughout the delivery of a medicament. In some embodiments, the injector 100 includes two or more stages. The first stage of the injector 100 may decrease in force as it nears the end of its delivery, thereby slowing the delivery of a medicament. The second stage of the injector 100 may activate after the first stage ends its delivery (e.g., when the first stage no longer causes the medicament to be expelled from the injector 100). The second stage of the injector 100 may activate before the first stage ends its delivery. For example, the first stage and the second stage of the injector 100 may both be active at the same time. This configuration may enhance the reliability of the injection process and maintain the required force for accurate dosage, thereby contributing to the overall safety and effectiveness of the device.

Referring to FIG. 1, the injector 100 may be an autoinjector or another handheld medical device designed for administration of a medicament. The injector 100 may include a housing 102 having a proximal end 104 and a distal end 106 extending along a longitudinal axis A_{L}. The housing 102 may be shaped and sized to be held in the hand of a user. The housing 102 may be held in one hand to facilitate self-administration of the medicament.

Referring to FIG. 1, the housing 102 may include a window 108 extending therethrough. The window 108 may be a generally oval shape. In some embodiments, the window 108 is a rectangular shape. In some embodiments, the housing 102 does not have a window 108. The window 108 may allow a user to inspect the medicament in the injector 100 before, during, or after use.

Referring to FIG. 1, the injector 100 may include a needle guard 112 extending from the distal end 106 of the housing 102. The needle guard 112 may be retractable to expose a needle when the needle guard 112 is pressed against an injection site. The injector 100 may include a trigger mechanism configured to activate the energy system 124, as described in greater detail below. The needle guard 112 may be a component of the trigger mechanism. While a needle guard 112 is shown, it should be appreciated that an alternative trigger mechanism may be implemented in the injector 100. In some embodiments, the trigger mechanism may utilize button activation, a twist-and-click mechanism, a push-and-hold button, a safety capsule release, pressure activation, skin-sensing activation, or any other suitable trigger mechanism for use in a handheld injector.

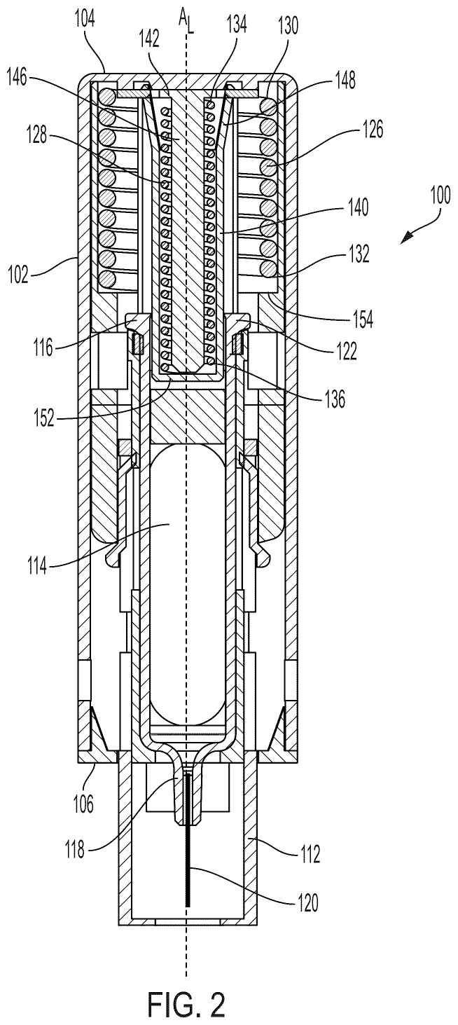

Referring to FIGS. 2-4, 6 and 7, the injector 100 may include a medicament container 114 containing a medicament disposed therein. The medicament container 114 may define a volume therein. The medicament container 114 may contain a volume of a medicament in the volume defined therein. The medicament container 114 may be fixed relative to the housing 102. As used herein, the medicament may comprise drugs, biologics, solutions, gels, suspensions or other substances that may be delivered via a syringe or needle, and such terms may be used interchangeably as appearing in the specification and claims.

The medicament container 114 may be a prefilled syringe. In some embodiments, the medicament container 114 may be an insertable medicament cartridge. In some embodiments, the medicament container 114 may be filled using a vial-and-needle or other appropriate filling method. In one embodiment, the medicament container 114 is one of a prefilled cartridge, prefilled staked needle syringe, vial, or other injectable fluid containing vessel.

The volume of the medicament may be at least 5 mL. In some embodiments, the volume of the medicament is at least 1 mL, at least 2 mL, at least 3 mL, at least 4 mL, at least 5 mL, at least 6 mL, at least 7 mL, at least 8 mL, at least 9 mL, at least 10 mL, at least 15 mL, at least 20 mL, at least 25 mL, or at least 30 mL. In some embodiments, the volume of the medicament is between 1 mL and 30 mL, between 2 mL and 25 mL, between 3 mL and 20 mL, between 4 mL and 15 mL, between 5 mL and 10 mL, between 6 mL and 9 mL, or between 7 mL and 8 mL.

The medicament may have a viscosity of less than 10 cPs. In some embodiments, the medicament has a viscosity of less than 1 cPs, less than 2 cPs, less than 3 cPs, less than 4 cPs, less than 5 cPs, less than 6 cPs, less than 7 cPs, less than 8 cPs, less than 9 cPs, less than 10 cPs, less than 11 cPs, less than 12 cPs, less than 13 cPs, less than 14 cPs, less than 15 cPs, less than 16 cPs, less than 17 cPs, less than 18 cPs, less than 19 cPs, or less than 20 cPs. In some embodiments, the medicament has a viscosity of between 1 cPs and 20 cPs, between 2 cPs and 19 cPs, between 3 cPs and 18 cPs, between 4 cPs and 17 cPs, between 5 cPs and 16 cPs, between 6 cPs and 15 cPs, between 7 cPs and 14 cPs, between 8 cPs and 13 cPs, between 9 cPs and 12 cPs, or between 10 cPs and 11 cPs.

Referring to FIGS. 2-4, 6 and 7, the medicament container 114 may have a proximal portion 116 and a distal portion 118 opposite the proximal portion 116 extending along a longitudinal axis A_{L}. A needle 120 may be coupled to and extending from the distal portion 118 of the medicament container 114. The needle 120 may be a staked needle. In some embodiments, the needle 120 may be coupled to the medicament container 114 prior to use. The needle 120 may be in fluid communication with the volume defined in the medicament container 114.

The needle 120 may be a 25 gauge needle. In some embodiments, the needle 120 is a 20 gauge needle, a 21 gauge needle, a 22 gauge needle, a 23 gauge needle, a 24 gauge needle, a 25 gauge needle, a 26 gauge needle, a 27 gauge needle, a 28 gauge needle, a 29 gauge needle, or a 30 gauge needle. In some embodiments, the needle 120 is between a 20 gauge and 30 gauge needle, between a 21 gauge and 29 gauge needle, between a 22 gauge and 28 gauge needle, between a 23 gauge and 27 gauge needle, or between a 24 gauge and 26 gauge needle.

The needle 120 may be a ½″ needle. In some embodiments, the needle 120 is a 5/16″ needle, a ⅜″ needle, a ½″ needle, a 9/16″ needle, a ⅝″ needle, a 11/16″ needle, a ¾″ needle, a 13/16″ needle, a ⅞″ needle, a 15/16″ needle, or a 1″ needle. In some embodiments, the needle 120 is between a 5/16″ and a 1″ needle, between a ⅜″ and a 15/16″ needle, between a ½″ and a ⅞″ needle, between a 9/16″ and a 13/16″ needle, or between a ⅝″ and a ¾″ needle.

Referring to FIGS. 2-4, 6 and 7, the medicament container 114 may include a flange 122. The flange 122 may extend radially outward from the proximal portion 116 of the medicament container 114. The flange 122 may be a generally circular member that extends around the circumference of the medicament container 114. In some embodiments, the flange 122 extends around only a portion of the circumference of the medicament container 114. The flange 122 may help to prevent the medicament container 114 from moving distally along the longitudinal axis A_{L} relative to the housing 102 when the energy system 124 is activated.

Referring to FIGS. 2-4, 6 and 7, the injector 100 may include an energy system 124. The energy system 124 may consist of one or more energy source. The energy system 124 may include a first energy source 126 and a second energy source 128. In some embodiments, the energy system 124 comprises the first energy source 126 and the second energy source 128. The first energy source 126 and the second energy source 128 may be compression springs; however, other suitable energy sources can be used, such as elastomer or compressed-gas springs, gas generators, or other suitable energy storage members.

Referring to FIGS. 2-4, 6 and 7, the second energy source 128 may be at least partially nested in the first energy source 126. The first energy source 126 may have a diameter of approximately 27 mm. In some embodiments, the first energy source 126 has a diameter of approximately 20 mm, approximately 21 mm, approximately 22 mm, approximately 23 mm, approximately 24 mm, approximately 25 mm, approximately 26 mm, approximately 27 mm, approximately 28 mm, approximately 29 mm, approximately 30 mm, approximately 31 mm, approximately 32 mm, approximately 33 mm, approximately 34 mm, or approximately 35 mm. In some embodiments, the first energy source 126 has a diameter of at least 20 mm, at least 21 mm, at least 22 mm, at least 23 mm, at least 24 mm, at least 25 mm, at least 26 mm, at least 27 mm, at least 28 mm, at least 29 mm, at least 30 mm, at least 31 mm, at least 32 mm, at least 33 mm, at least 34 mm, or at least 35 mm. In some embodiments, the first energy source 126 has a diameter between 20 mm and 35 mm, between 21 mm and 34 mm, between 22 mm and 33 mm, between 23 mm and 32 mm, between 24 mm and 31 mm, between 25 mm and 30 mm, between 26 mm and 29 mm, or between 27 mm and 28 mm.

The second energy source 128 may have a diameter of approximately 9 mm. In some embodiments, the second energy source 128 has a diameter of approximately 5 mm, approximately 5.5 mm, approximately 6 mm, approximately 6.5 mm, approximately 7 mm, approximately 7.5 mm, approximately 8 mm, approximately 8.5 mm, approximately 9 mm, approximately 9.5 mm, approximately 10 mm, approximately 10.5 mm, approximately 11 mm, approximately 11.5 mm, approximately 12 mm, approximately 12.5 mm, approximately 13 mm, approximately 13.5 mm, or approximately 14 mm. In some embodiments, the second energy source 128 has a diameter of at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, at least 10 mm, at least 10.5 mm, at least 11 mm, at least 11.5 mm, at least 12 mm, at least 12.5 mm, at least 13 mm, at least 13.5 mm, or at least 14 mm. In some embodiments, the second energy source 128 has a diameter between 5.5 mm and 14 mm, between 6 mm and 13.5 mm, between 6.5 mm and 13 mm, between 7 mm and 12.5 mm, between 7.5 mm and 12 mm, between 8 mm and 11.5 mm, between 8.5 mm and 11 mm, between 9 mm and 10.5 mm, or between 9.5 mm and 10 mm.

The first energy source 126 may have a proximal side 130 and a distal side 132 extending along the longitudinal axis A_{L}. The second energy source 128 may have a proximal side 134 and a distal side 136 extending along the longitudinal axis A_{L}. In some embodiments, the first energy source 126 and the second energy source 128 are connected in series. In some embodiments, the distal side 132 of the first energy source 126 engages the proximal side 134 of the second energy source 128.

Referring to FIGS. 2-4, 6 and 7, the injector 100 may include a first ram 138 engageable with the first energy source 126 and a second ram 140 engageable with the second energy source 128. The second ram 140 may be at least partially nested within the first ram 138.

The first ram 138 may be a generally cylindrical member and may be movable relative to the housing 102. The first ram 138 may be shaped and sized with a similar cross section to that of the housing 102 to allow the first ram 138 to slide along an inner surface of the housing 102. The first ram 138 may be disposed between the housing 102 and the medicament container 114. The first ram 138 may have a proximal surface 142. The proximal surface 142 may include at least one ram aperture 144 extending therethrough. The ram aperture 144 may be shaped and sized to allow a member to pass therethrough. The proximal surface 142 may have a central post 146 extending distally therefrom. The central post 146 may be a generally cylindrical member. The central post 146 may be shaped and sized to extend through the second energy source 128. The central post 146 may guide the second energy source 128 to ensure that the second energy source 128 travels the longitudinal axis A_{L} when the second energy source 128 is activated. The first ram 138 may include a shelf 154 extending radially inward. The shelf 154 may be spaced apart from the proximal surface 142.

The second ram 140 may be a generally cylindrical member and may be movable relative to the housing 102 and the first ram 138, as described in greater detail below. The second ram 140 may include a distal surface 152. The second ram 140 may include at least one flexible arm 148 extending therefrom. The flexible arm 148 may extend proximally from the second ram 140. The second ram 140 may include two flexible arms 148 spaced apart from each other.

Referring to FIGS. 2-4, 6 and 7, the first energy source 126 may engage the housing 102 and the first ram 138. The first energy source 126 may engage the proximal end 104 of the housing 102 and the shelf 154 of the first ram 138. The second energy source 128 may engage the first ram 138 and the second ram 140. The second energy source 128 may engage the proximal surface 142 of the first ram 138 and the distal surface 152 of the second ram 140.

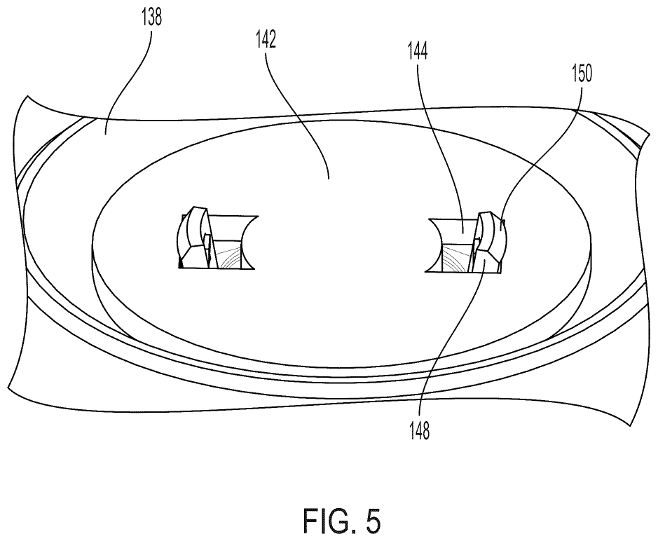

Referring to FIG. 5, the flexible arm 148 may extend through the ram aperture 144. The flexible arm 148 may include an engagement feature 150 radially extending therefrom. The engagement feature 150 may releasably couple the second ram 140 to the first ram 138.

The first energy source 126 may produce at least 44 lbf of force. In some embodiments, the first energy source 126 produces at least 20 lbf of force, at least 25 lbf of force, at least 30 lbf of force, at least 35 lbf of force, at least 40 lbf of force, at least 45 lbf of force, at least 50 lbf of force, at least 55 lbf of force, at least 60 lbf of force, at least 65 lbf of force, or at least 70 lbf of force when compressed. In some embodiments, the first energy source 126 produces between 20 lbf of force and 70 lbf of force, between 25 lbf of force and 65 lbf of force, between 30 lbf of force and 60 lbf of force, between 35 lbf of force and 55 lbf of force, or between 40 lbf of force and 50 lbf of force.

The second energy source 128 may produce at least 14 lbf of force. In some embodiments, the second energy source 128 produces at least 5 lbf of force, at least 6 lbf of force, at least 7 lbf of force, at least 8 lbf of force, at least 9 lbf of force, at least 10 lbf of force, at least 11 lbf of force, at least 12 lbf of force, at least 13 lbf of force, at least 14 lbf of force, at least 15 lbf of force, at least 16 lbf of force, at least 17 lbf of force, at least 18 lbf of force, at least 19 lbf of force, at least 20 lbf of force, at least 21 lbf of force, at least 22 lbf of force, at least 23 lbf of force, at least 24 lbf of force, or at least 25 lbf of force. In some embodiments, the second energy source 128 produces between 5 lbf of force and 25 lbf of force, between 6 lbf of force and 24 lbf of force, between 7 lbf of force and 23 lbf of force, between 8 lbf of force and 22 lbf of force, between 9 lbf of force and 21 lbf of force, between 10 lbf of force and 20 lbf of force, between 11 lbf of force and 19 lbf of force, between 12 lbf of force and 18 lbf of force, between 13 lbf of force and 17 lbf of force, or between 14 lbf of force and 16 lbf of force.

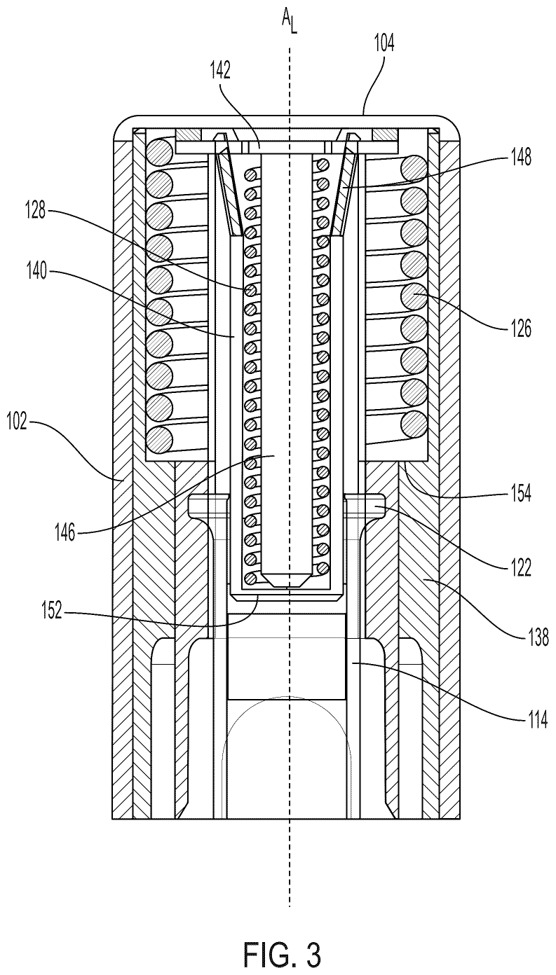

Referring to FIG. 3, the injector 100 is shown in a pre-firing configuration. In the pre-firing configuration, the first energy source 126 and the second energy source 128 are in compressed states. The first ram 138 may engage the proximal end 104 of the housing 102. In some embodiments, the first ram 138 is proximate the proximal end 104 of the housing 102 in the pre-firing configuration. The second ram 140 may be detachably coupled to the first ram 138.

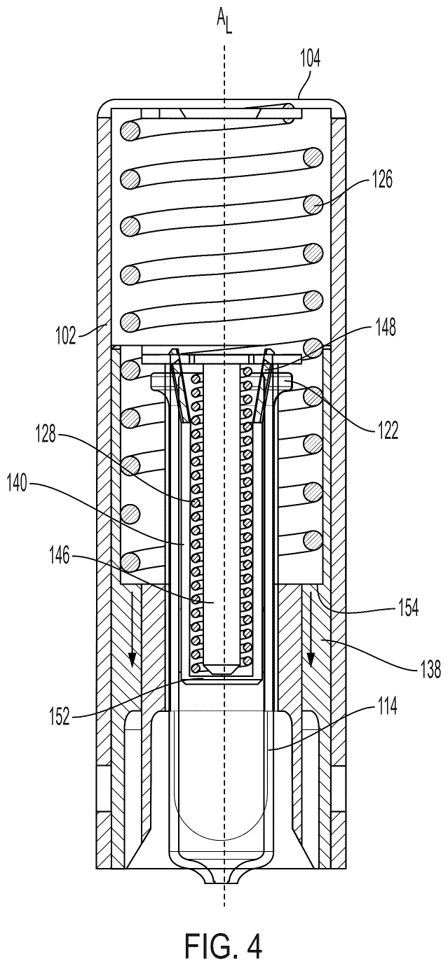

Referring to FIGS. 3-4, during a first triggering event, the first energy source 126 is configured to be activated before the second energy source 128. When the trigger mechanism (e.g., needle guard 112) activates the energy system 124, the first energy source 126 may move the first ram 138 distally along the longitudinal axis A_{L} relative to the housing 102. Movement of the first ram 138 may move the second ram 140 relative to the housing 102. The second ram 140 may move within the medicament container 114 to eject a first volume of the medicament. The first energy source 126 may extend outside of the medicament container 114 when activated. The second energy source 128 may extend inside of the medicament container 114 when activated.

Referring to FIG. 4, the injector 100 is shown in a first fired configuration. In the first fired configuration, the first ram 138 may engage the distal end 106 of the housing 102, thereby preventing the first ram 138 from moving distally relative to the housing 102. The second ram 140 may be at least partially disposed in the medicament container 114 in the first fired configuration. Activation of the first energy source 126 and movement of the first ram 138 along the longitudinal axis A_{L} relative to the housing 102 may cause engagement of the flexible arm 148 and the flange 122. The flexible arm 148 of the second ram 140 may engage the flange 122 of the medicament container 114, thereby urging the flexible arm 148 radially inward.

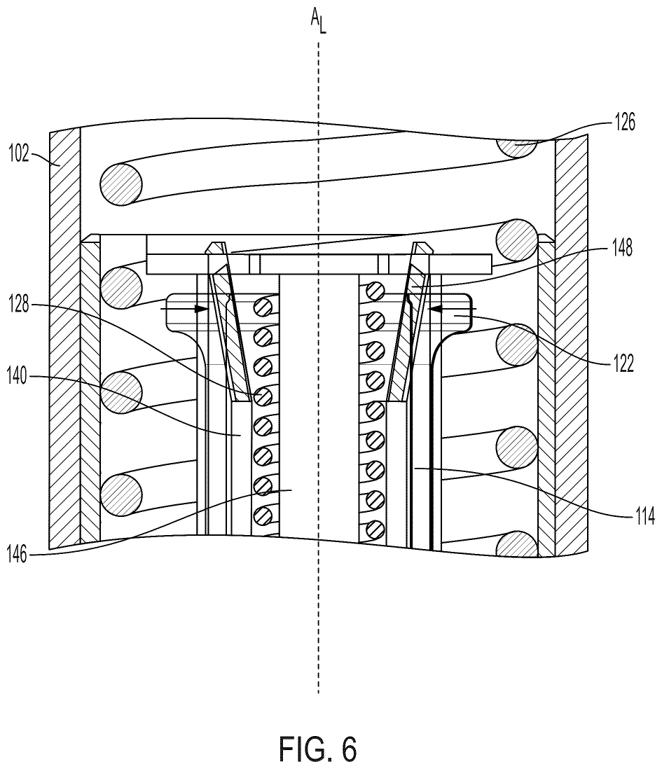

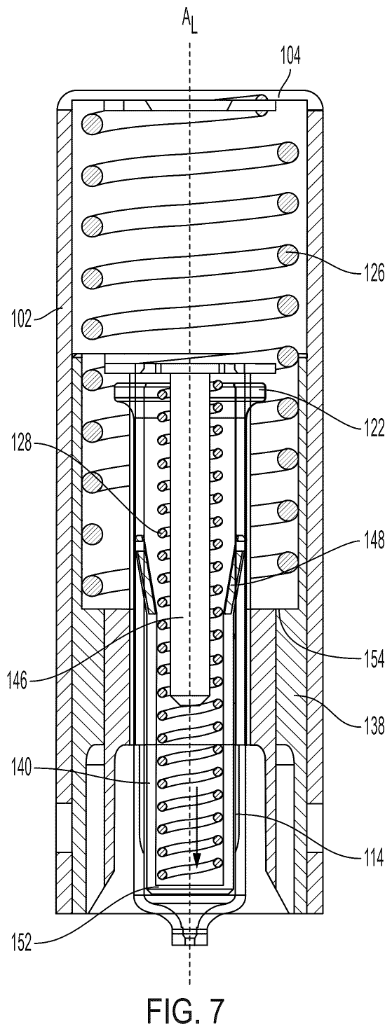

Referring to FIGS. 6-7, engagement of the flexible arm 148 and the flange 122 may disengage the engagement feature 150 of the flexible arm 148 from the ram aperture 144, thereby activating the second energy source 128. The second energy source 128 may urge the second ram 140 distally along the longitudinal axis A_{L} relative to the medicament container 114. The second ram 140 may be movable distally along the longitudinal axis A_{L} relative to the first ram 138. The second energy source 128 may be activated before the injector 100 is in the first fired configuration. The second ram 140 may be moved distally along the longitudinal axis A_{L} relative to the medicament container 114 by the first energy source 126 and the second energy source 128 simultaneously. In some embodiments, the second ram 140 may be moved distally along the longitudinal axis A_{L} relative to the medicament container 114 by the first energy source 126 and then the second energy source 128 in a sequence. In some embodiments, the first energy source 126 of the injector 100 may be stopped before the end of its delivery to avoid a decrease in force and subsequent decrease in the delivery rate of the medicament. In some embodiments, the second energy source 128 of the injector 100 may be stopped before the end of its delivery to avoid a decrease in force and subsequent decrease in the delivery rate of the medicament.

Referring to FIG. 7, the injector 100 is shown in a second fired configuration. In the second fired configuration, the second ram 140 may engage an end of the medicament container 114, thereby preventing the second ram 140 from moving distally relative to the medicament container 114. The second ram 140 may be fully disposed within the volume of the medicament container 114 in the second fired configuration.

The second ram 140 may be movable a first distance within the volume of the medicament container 114 when the first energy source 126 is activated. The second ram 140 may be movable a second distance when the second energy source 128 is activated. The first distance may be greater than the second distance.

The first distance may be approximately 33 mm when the first energy source 126 is activated. In some embodiments, the first distance is approximately 25 mm, approximately 26 mm, approximately 27 mm, approximately 28 mm, approximately 29 mm, approximately 30 mm, approximately 31 mm, approximately 32 mm, approximately 33 mm, approximately 34 mm, approximately 35 mm, approximately 36 mm, approximately 37 mm, approximately 38 mm, approximately 39 mm, or approximately 40 mm when the first energy source 126 is activated. In some embodiments, the first distance is at least 25 mm, at least 26 mm, at least 27 mm, at least 28 mm, at least 29 mm, at least 30 mm, at least 31 mm, at least 32 mm, at least 33 mm, at least 34 mm, at least 35 mm, at least 36 mm, at least 37 mm, at least 38 mm, at least 39 mm, or at least 40 mm when the first energy source 126 is activated. In some embodiments, the first distance is between 25 mm and 40 mm, between 26 mm and 39 mm, between 27 mm and 38 mm, between 28 mm and 37 mm, between 29 mm and 36 mm, between 30 mm and 35 mm, between 31 mm and 34 mm, or between 32 mm and 33 mm.

The second distance may be approximately 23 mm when the second energy source 128 is activated. In some embodiments, the second distance is approximately 15 mm, approximately 16 mm, approximately 17 mm, approximately 18 mm, approximately 19 mm, approximately 20 mm, approximately 21 mm, approximately 22 mm, approximately 23 mm, approximately 24 mm, approximately 25 mm, approximately 26 mm, approximately 27 mm, approximately 28 mm, approximately 29 mm, approximately 30 mm, approximately 31 mm, approximately 32 mm, approximately 33 mm, approximately 34 mm, or approximately 35 mm when the second energy source 128 is activated. In some embodiments, the second distance is at least 15 mm, at least 16 mm, at least 17 mm, at least 18 mm, at least 19 mm, at least 20 mm, at least 21 mm, at least 22 mm, at least 23 mm, at least 24 mm, at least 25 mm, at least 26 mm, at least 27 mm, at least 28 mm, at least 29 mm, at least 30 mm, at least 31 mm, at least 32 mm, at least 33 mm, at least 34 mm, or at least 35 mm when the second energy source 128 is activated. In some embodiments, the second distance is between 15 mm and 35 mm, between 16 mm and 34 mm, between 17 mm and 33 mm, between 18 mm and 32 mm, between 19 mm and 31 mm, between 20 mm and 30 mm, between 21 mm and 29 mm, between 22 mm and 28 mm, between 23 mm and 27 mm, or between 24 mm and 26 mm when the second energy source 128 is activated.

The energy system 124 may be configured to eject the volume of the medicament from the medicament container 114 in less than 30 seconds. In some embodiments, the energy system 124 is configured to eject the volume of the medicament from the medicament container 114 in less than 10 seconds, less than 15 seconds, less than 20 seconds, less than 25 seconds, less than 30 seconds, less than 35 seconds, less than 40 seconds, or less than 45 seconds. In some embodiments, the energy system 124 is configured to eject the volume of the medicament from the medicament container 114 between 10 seconds and 45 seconds, between 15 seconds and 40 seconds, between 20 seconds and 35 seconds, or between 25 seconds and 30 seconds.

The energy system 124 may be configured to eject the volume of the medicament from the medicament container 114 at a rate of approximately 0.25 mL/sec. In some embodiments, the energy system 124 is configured to eject the volume of the medicament from the medicament container 114 at a rate of approximately 0.1 mL/sec, approximately 0.125 mL/sec, approximately 0.15 mL/sec, approximately 0.175 mL/sec, approximately 0.2 mL/sec, approximately 0.225 mL/sec, approximately 0.25 mL/sec, approximately 0.275 mL/sec, approximately 0.3 mL/sec, approximately 0.325 mL/sec, approximately 0.35 mL/sec, approximately 0.375 mL/sec, approximately 0.4 mL/sec, approximately 0.425 mL/sec, or approximately 0.45 mL/sec. In some embodiments, the energy system 124 is configured to eject the volume of the medicament from the medicament container 114 at a rate of at least 0.1 mL/sec, at least 0.125 mL/sec, at least 0.15 mL/sec, at least 0.175 mL/sec, at least 0.2 mL/sec, at least 0.225 mL/sec, at least 0.25 mL/sec, at least 0.275 mL/sec, at least 0.3 mL/sec, at least 0.325 mL/sec, at least 0.35 mL/sec, at least 0.375 mL/sec, at least 0.4 mL/sec, at least 0.425 mL/sec, or at least 0.45 mL/sec. In some embodiments, the energy system 124 is configured to eject the volume of the medicament from the medicament container 114 at a rate between 0.1 mL/sec and 0.45 mL/sec, between 0.125 mL/sec and 0.425 mL/sec, between 0.15 mL/sec and 0.4 mL/sec, between 0.175 mL/sec and 0.375 mL/sec, between 0.2 mL/sec and 0.35 mL/sec, between 0.225 mL/sec and 0.325 mL/sec, or between 0.25 mL/sec and 0.3 mL/sec.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether or not “about” is used in conjunction therewith. It should also be appreciated that the term “about”, as used herein in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

What is claimed is:

1. An injector comprising:
a housing extending along a longitudinal axis, the housing having a proximal end and a distal end;
a medicament container defining a volume therein;
a needle coupled to and extending from a distal end of the medicament container; and
an energy system having a first energy source and a second energy source,
wherein the first energy source is configured to be activated before the second energy source.

2. The injector of claim 1 further comprising:
a first ram engageable with the first energy source and a second ram engageable with the second energy source.

3. The injector of claim 2, wherein movement of the first ram moves the second ram relative to the housing.

4. The injector of claim 3, wherein the second ram is detachably coupled to the first ram, and
wherein the second ram further includes a flexible arm extending therefrom, the flexible arm releasably engageable with the first ram.

5. The injector of claim 4, wherein the medicament container includes a flange extending radially outward from a proximal end,
wherein activation of the first energy source and movement of the first ram along the longitudinal axis relative to the housing causes engagement of the flexible arm and the flange, and
wherein the flange disengages the flexible arm from the first ram, thereby activating the second energy source.

6. The injector of claim 5, wherein the first ram includes a ram aperture extending therethrough configured to receive the flexible arm, and
wherein the flexible arm includes an engagement feature extending radially therefrom, the engagement feature engageable with the ram aperture to releasably couple the second ram to the first ram.

7. The injector of claim 6, wherein engagement of the flexible arm and the flange disengages the engagement feature of the flexible arm from the ram aperture.

8. The injector of claim 5, wherein the second energy source urges the second ram distally along the longitudinal axis relative to the medicament container.

9. The injector of claim 8, wherein the second ram is movable along the longitudinal axis relative to the first ram.

10. The injector of claim 2, wherein the second ram is movable a first distance within the volume of the medicament container when the first energy source is activated and a second distance when the second energy source is activated.

11. The injector of claim 10, wherein the first distance is greater than the second distance.

12. The injector of claim 2, wherein the first energy source engages the housing and the first ram.

13. The injector of claim 2, wherein the second energy source engages the first ram and the second ram.

14. The injector of claim 1, wherein the second energy source is at least partially nested in the first energy source.

15. The injector of claim 14, wherein the first energy source extends outside of the medicament container when activated and wherein the second energy source extends inside of the medicament container when activated.

16. The injector of claim 1, wherein the medicament container contains a volume of a medicament.

17. The injector of claim 16, wherein the energy system is configured to eject the volume of the medicament from the medicament container in less than 30 seconds.

18. The injector of claim 1, wherein the first energy source and the second energy source are connected in series.

19. The injector of claim 1 further comprising:
a trigger mechanism configured to activate the first energy source.

20. An injector comprising:
a housing extending along a longitudinal axis, the housing having a proximal end and a distal end;
a medicament container defining a volume therein;
a needle coupled to and extending from a distal end of the medicament container;
an energy system having a first energy source and a second energy source; and
a first ram engageable with the first energy source and a second ram engageable with the second energy source.
wherein the first energy source and the second energy source are connected in series, and
wherein the second energy source is at least partially nested in the first energy source.

21. An injector comprising:
a housing extending along a longitudinal axis, the housing having a proximal end and a distal end;
a medicament container defining a volume therein and containing a volume of a medicament, the medicament container including a flange extending radially outward from a proximal end;
a needle coupled to and extending from a distal end of the medicament container;
an energy system having a first spring and a second spring connected in series;
a first ram engageable with the first spring and a second ram detachably coupled to the first ram and engageable with the second spring, the second ram including a flexible arm extending therefrom, the flexible arm releasably engageable with the first ram; and
a trigger mechanism configured to activate the first spring,
wherein the first spring is configured to be activated before the second spring,
wherein movement of the first ram moves the second ram relative to the housing,
wherein activation of the first spring and movement of the first ram along the longitudinal axis relative to the housing causes engagement of the flexible arm and the flange,
wherein the flange disengages the flexible arm from the first ram, thereby activating the second spring,
wherein the second spring urges the second ram distally along the longitudinal axis relative to the medicament container and relative to the first ram,
wherein the second ram is movable a first distance within the volume of the medicament container when the first spring is activated and a second distance when the second spring is activated, where the first distance is greater than the second distance,
wherein the first spring engages the housing and the first ram and the second spring engages the first ram and the second ram, and
wherein the second spring is at least partially nested in the first spring and where the first spring extends outside of the medicament container when activated and where the second spring extends inside of the medicament container when activated.
